(19) United States
(12) Patent Application Publication
(10) Pub. No.: US 2022/0347184 A1
(43) Pub. Date: Nov. 3, 2022

(54) SKIN-PENETRATING FORMULATION OF TAUROLIDINE

(71) Applicant: CorMedix Inc., Berkeley Heights, NJ (US)

(72) Inventors: Bruce Reidenberg, Rye, NY (US); Robert DiLuccio, Viera, FL (US)

(21) Appl. No.: 17/675,639

(22) Filed: Feb. 18, 2022

Related U.S. Application Data
(63) Continuation of application No. 15/287,822, filed on Oct. 7, 2016, now abandoned.
(60) Provisional application No. 62/238,167, filed on Oct. 7, 2015.

(51) Int. Cl. A61K 31/549; A61K 9/00; A61K 9/51; A61K 47/12; A61K 9/06; A61K 47/36

(52) U.S. Cl. CPC A61K 31/549; A61K 9/0014; A61K 9/5161; A61K 47/12; A61K 9/06; A61K 47/36

(57) ABSTRACT

A composition comprising: hydrolysable taurolidine; and a hydrolysable lipophilic excipient; wherein the hydrolysable taurolidine is contained within the hydrolysable lipophilic excipient.

### REFERENCE TO PENDING PRIOR PATENT APPLICATION

This patent application claims benefit of pending prior U.S. Provisional Patent Application Ser. No. 62/238,167, filed Oct. 7, 2015 by CorMedix Inc. and Bruce Reidenberg et al. for SKIN-PENETRATING FORMULATION OF TAUROLIDINE (Attorney's Docket No. CORMEDIX-13 PROV), which patent application is hereby incorporated herein by reference.

### FIELD OF THE INVENTION

This invention relates to medical treatments in general, and more particularly to medical treatments utilizing taurolidine.

### BACKGROUND OF THE INVENTION

Excipients designed to improve skin penetration of water-soluble drugs is a well-established field. The usual goal of applying excipients to the skin is to induce a temporary break in the barrier function of the skin so that a sufficient amount of a drug can be systemically absorbed using the subdermal venous plexus.

Taurolidine is a well-known antimicrobial with a published mechanism of action and antimicrobial spectrum. Taurolidine is unstable in circulation and therefore has not been successfully developed for systemic infections. Taurolidine has demonstrated efficacy in local application for peritonitis and for the prevention of infection when infused as a catheter-lock solution.

### SUMMARY OF THE INVENTION

Taurolidine is an antimicrobial with a broad spectrum of activity due to its hydrolysis products (i.e., methylol groups). The use of taurolidine in skin infections is impaired by the breakdown of the taurolidine at the skin surface. The present invention provides a specialized taurolidine formulation which is designed to maintain taurolidine stability during the skin penetration process. Once this specialized taurolidine formulation has facilitated passage of the taurolidine through the stratum corneum, lucidum, and spinosum layers of the skin (see FIGS. 1 and 2), the taurolidine in the specialized taurolidine formulation is exposed to the anatomy and hydrolysis to the active moieties of taurolidine (i.e., methylol groups) can occur, whereby to treat skin infections and to prevent skin infections. This specialized taurolidine formulation comprises lipid-soluble excipients that are hydrolysable by enzymes in the stratum granulosum or the dermis layers of the skin. Such lipid-soluble excipients include small peptides with lipophilic side chains and fatty acid esters.

Note that the present invention is not directed to the use of an excipient to promote systemic absorption of the taurolidine - rather, it is designed to deliver taurolidine, a hydrolysable composition, to the site of action where the taurolidine can hydrolyze into the active moieties of taurolidine (i.e., methylol groups) to achieve local antimicrobial effects.

If desired, the specialized taurolidine formulation may also comprise an emulsion, with the taurolidine and the lipid-soluble excipient being suspended in the emulsion.

A further refinement of the present invention includes creating nanoparticles with taurolidine centers and lipophilic exteriors suspended in an emulsion.

The specialized taurolidine formulation is intended to be administered once or twice daily until the skin is healed. This product can be for local skin infections or as a part of comprehensive burn treatment. Optionally, skin penetrant enhancers (e.g., additional types of lipid-soluble excipients) may be incorporated into the specialized taurolidine formulation to allow for enhanced delivery of the taurolidine through the skin.

In one preferred form of the present invention, there is provided a composition comprising:

hydrolysable taurolidine; and

a hydrolysable lipophilic excipient;

wherein the hydrolysable taurolidine is contained within the hydrolysable lipophilic excipient.

In another preferred form of the present invention, there is provided a novel pharmaceutical composition comprising:

(i) a therapeutically-effective amount of taurolidine or a pharmaceutically-acceptable salt thereof;

(ii) an effective penetration-enhancing hydrolysable lipophilic excipient which facilitates passage of the taurolidine through the outer layers of the skin and temporarily protects the taurolidine from premature hydrolization to active moieties as the taurolidine passes through the outer layers of the skin; and

(iii) a suitable pharmaceutical carrier.

In another preferred form of the present invention, there is provided a method for treating a patient, the method comprising:

applying a composition to the skin of a patient, the composition comprising:
hydrolysable taurolidine; and
a hydrolysable lipophilic excipient;
wherein the hydrolysable taurolidine is contained within the hydrolysable lipophilic excipient; and

leaving the composition on the skin of the patient long enough for the hydrolysable lipophilic excipient to facilitate passage of the composition through the skin and, as the composition passes through the skin, the lipophilic excipient is hydrolyzed, exposing the hydrolysable taurolidine to the anatomy, whereupon the taurolidine hydrolyzes into its active moieties so as to provide local antimicrobial effects.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing one form of the specialized taurolidine formulation of the present invention penetrating the skin of a patient;

FIG. 2 is a schematic view showing another form of the specialized taurolidine formulation of the present invention penetrating the skin of a patient; and

FIG. 3 is a graph showing the activity of taurolidine-loaded hydrogels against biofilm on a Pig Skin Explant Model.

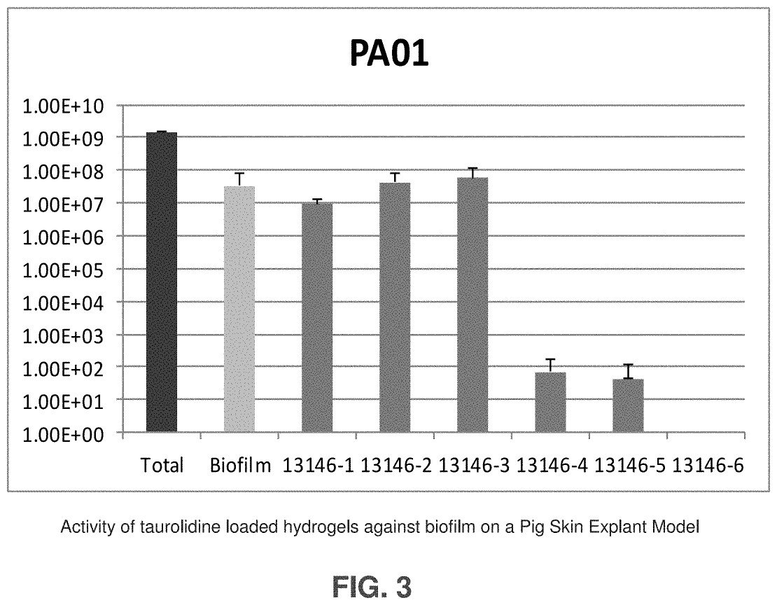

### DETAILED DESCRIPTION OF THE PREFERRED EMBODIMENTS

The present invention comprises the provision and use of a novel skin-penetrating formulation of taurolidine designed to deliver the taurolidine to an internal infection site, whereby to treat skin infections and to prevent skin infections, e.g., such as in burn victims.

Transdermal drug delivery is distinguished from topical drug delivery by the fact that, while a transdermal formulation is specifically designed to provide a predictable and therapeutically significant rate of delivery of the drug to the systemic circulation, a topical formulation is specifically designed to provide a therapeutic effect to only the local area where the drug is applied. Furthermore, topical formulations are often designed to prevent any systemic delivery of the drug in order to minimize side effects from the drug. However, where the topical delivery of a drug results in systemic absorption, the amount of drug delivery to the circulation is variable and uncontrolled.

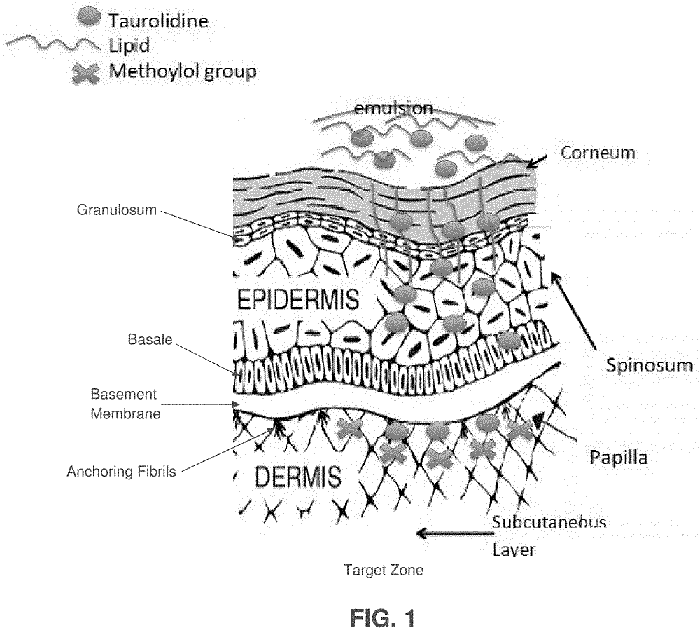

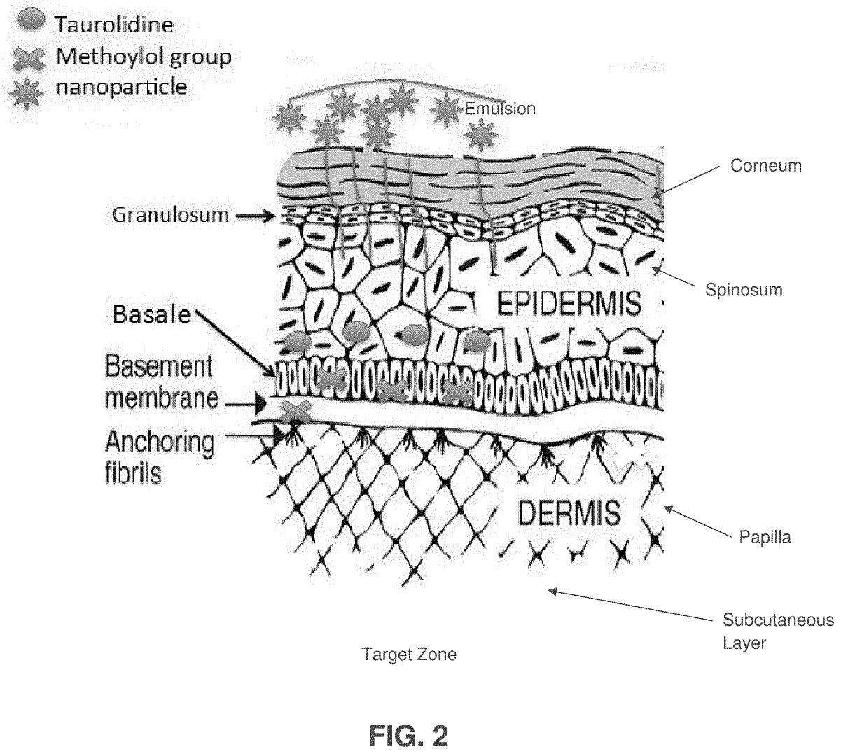

The goal of the present invention is the localized delivery (i.e., topical drug delivery) of taurolidine that penetrates and resides in several layers of the skin including the epidermis, dermis, and subcutaneous layers of the skin. See FIGS. 1 and 2. Although some of the taurolidine may end up in systemic circulation, the present invention is designed so that the bulk of the taurolidine remains localized to the point of application.

The skin is an excellent barrier to the penetration of many foreign substances. The feasibility of using topical delivery to pass taurolidine through the skin requires that a therapeutic quantity, and/or rate of delivery, of taurolidine be delivered through the skin. Normally this cannot be achieved with taurolidine, due to the substantial barrier properties of the skin. However, topical delivery of taurolidine can be made possible if the skin is made more permeable to the taurolidine (and/or if the taurolidine is protected from premature hydrolysis of the taurolidine in the outer layers of the skin). This may be accomplished by modifying the taurolidine permeability of the skin and/or by using a “vehicle” to carry the taurolidine through the skin, whereby to facilitate topical delivery of the taurolidine.

Factors that determine the permeability of the skin to a particular drug include drug diffusivity through the skin, vehicle/skin drug partitioning, and drug concentration in the vehicle. In addition, certain materials used as adjuvants in vehicles may affect the characteristics of the skin barrier and thus alter the permeability of the skin to the drug. Such materials are referred to as skin penetration enhancers. These skin penetration enhancers are important in the optimization of topical drug delivery because of the necessity for the maximization of penetration rates and the minimization of lag times in the drug penetration through the skin.

The permeability of the skin to a drug is influenced by a combination of physico-chemical parameters for both the drug and the vehicle, as discussed above. Thus, effective topical delivery of a particular drug requires the selection of an appropriate vehicle. The optimum vehicle for one drug may not be effective for topical delivery of another drug since the properties of the vehicle and the drug must be matched to ensure a therapeutic rate of drug delivery through the skin.

The present invention relates to a novel pharmaceutical composition that provides topical delivery of therapeutically-effective amounts of taurolidine to desired regions of mammalian skin.

In one preferred form of the present invention, the novel pharmaceutical composition comprises:

a therapeutically-effective amount of hydrolysable taurolidine (e.g., taurolidine or a pharmaceutically-acceptable salt thereof, sometimes referred to herein as simply “the taurolidine”); and

an effective penetration-enhancing amount of a hydrolysable lipophilic excipient (e.g., at least one of a saturated fatty alcohol or fatty acid of 8-15 carbon atoms or of an unsaturated fatty alcohol or fatty acid of 8-18 carbon atoms).

If desired, the novel pharmaceutical composition may also comprise a suitable pharmaceutical carrier (e.g., an emulsion) for carrying the therapeutically-effective amount of hydrolysable taurolidine and the effective penetration-enhancing amount of a hydrolysable lipophilic excipient to the skin of a patient.

The hydrolysable lipophilic excipient of the novel pharmaceutical composition protects the taurolidine from hydrolysis while the taurolidine is diffusing through the superficial layers of the skin, then releases the taurolidine at the site of infection in the stratum granulosum or the dermis, whereupon the taurolidine hydrolyzes to its active moieties (i.e., methylol groups), whereby to treat the infection (or to prevent infection). This selective delivery of the taurolidine is accomplished with the lipophilic excipient acting on the tissue to facilitate passage of the composition through the tissue and with the lipophilic excipient also acting to shield the hydrolysable taurolidine from premature hydrolysis in the outer layers of the skin. The lipophilic excipient is hydrolysable by tissue enzymes in the deeper layers of skin. The lipophilicity of the hydrolysable excipient allows the “protected” taurolidine (contained within the hydrolysable excipient) to pass through inter-cellular hydrophobic channels in the stratum corneum through to the stratum granulosum and, potentially, on to the dermis. Once deep in the stratum granulosum (or the dermis), local extracellular enzymes degrade the protective hydrophobic excipient and expose the taurolidine to local hydrolysis, thereby creating the active moieties (i.e., methylol groups) which treat the infection.

In one form of the invention, a mass of the therapeutically-effective amount of hydrolysable taurolidine is mixed into a mass of the effective penetration-enhancing amount of a hydrolysable lipophilic excipient so that the hydrolysable lipophilic excipient covers the hydrolysable taurolidine as the mixture penetrates the superficial layers of the skin, protecting the hydrolysable taurolidine from hydrolyzing in the superficial layers of the skin. Thereafter, the hydrolysable taurolidine is exposed to the tissue of the patient in the deeper layers of the skin, where the hydrolysable taurolidine is hydrolyzed to its active moieties (i.e., methylol groups), whereby to provide local antimicrobial effect. See FIG. 1.

In another form of the invention, the hydrolysable taurolidine is encapsulated within the hydrolysable lipophilic excipient so as to form nanoparticles (comprising taurolidine centers and lipophilic exteriors) so that the hydrolysable lipophilic excipient covers the hydrolysable taurolidine as the mixture penetrates the superficial layers of the skin, protecting the hydrolysable taurolidine from hydrolyzing in the superficial layers of the skin. Thereafter, the hydrolysable taurolidine is exposed to the tissue of the patient in the deeper layers of the skin, where the hydrolysable taurolidine is hydrolyzed to its active moieties (i.e., methylol groups), whereby to provide local antimicrobial effect. See FIG. 2.

Thus, in either form of the invention, the hydrolysable taurolidine is covered by a hydrolysable lipophilic excipient, with either the hydrolysable taurolidine being mixed into a mass of a hydrolysable lipophilic excipient or with the hydrolysable taurolidine being encapsulated by a hydrolysable lipophilic excipient (i.e., so as to form nanoparticles). When the mixture or nanoparticles are applied to the skin, the hydrolysable lipophilic excipient facilitates passage of the mixture or nanoparticles through the skin. As the mixture or nanoparticles pass through the skin, the lipophilic excipient is hydrolyzed, exposing the hydrolysable taurolidine to the anatomy, whereupon the taurolidine hydrolyzes into its active moieties (i.e., methylol groups) which treat the infection (or prevent infection).

In one preferred form of the invention, the mixture or nanoparticles are delivered to the skin in a suitable pharmaceutical carrier, e.g., an emulsion.

In one form of the invention, the hydrolysable lipophilic excipient comprises at least one of a saturated fatty alcohol or fatty acid of 8-15 carbon atoms or an unsaturated fatty alcohol or fatty acid of 8-18 carbon atoms.

For the purposes of the present disclosure, the terms “fatty alcohol” and/or “fatty acid” are meant to mean any saturated fatty acid or fatty alcohol having from 8 to 15 carbon atoms or any unsaturated fatty acid or fatty alcohol having from 8 to 18 carbon atoms which is effective in enhancing the penetration of taurolidine through desired regions of the mammalian skin.

It should also be appreciated that the present invention may utilize any combination of fatty acids and/or fatty alcohols having the above-specified number of carbon atoms, which is effective in enhancing topical taurolidine penetration. Preferred penetration-enhancing fatty acids and fatty alcohols are those with 10-15 carbon atoms or any mixture thereof. Especially preferred penetration-enhancing fatty acids and fatty alcohols are those with 14 carbon atoms such as myristic acid and myristyl alcohol. It should be understood that the terms “penetration enhancer” and/or “fatty acid” and/or “fatty alcohol” are used interchangeably throughout the present disclosure.

And in one form of the invention, the hydrolysable lipophilic excipient comprises small peptides with lipophilic side chains and fatty acid esters. The small peptides may comprise a high percentage of valine, leucine, proline, phenylalanine, tryptophan and/or leucine-enkephalin. The fatty acid esters may include 10-15 carbon saturated and unsaturated fatty esters. The fatty acid esters may include compositions comprising diglycerides, triglycerides, and glycerol monostearate.

By the term “suitable pharmaceutical carrier” is meant any non-toxic pharmaceutically-suitable vehicle, e.g., an emulsion. In one preferred form of the invention, the suitable pharmaceutical carrier may comprise any polar protic solvent with a molecular weight of less than 600. Suitable carriers include propylene glycol, polyethylene glycol, petrolatum, glycerin, polyvinylpyrrolidone and hyaluronic acid. Propylene glycol is a preferred carrier or vehicle, and any other carriers that may be used are then considered to be excipients.

All starting materials useful in making the pharmaceutical compositions of the present invention are known to those skilled in the art.

Thus, the present invention comprises the provision and use of a topical formulation comprising taurolidine which is designed to deliver the taurolidine to an internal infection site, whereby to treat skin infections and to prevent skin infections, e.g., such as in burn victims.

In one preferred form of the invention, there is provided a novel pharmaceutical composition which comprises:

(i) a therapeutically-effective amount of taurolidine or a pharmaceutically-acceptable salt thereof (sometimes referred to herein as “the taurolidine”);

(ii) an effective penetration-enhancing hydrolysable lipophilic excipient (sometimes referred to herein as “the hydrolysable excipient” or “the lipophilic excipient”) which facilitates passage of the taurolidine through the outer layers of the skin and temporarily protects the taurolidine from premature hydrolization to its active moieties (i.e., methylol groups) as the taurolidine passes through the outer layers of the skin; and

(iii) a suitable pharmaceutical carrier (e.g., an emulsion).

In one preferred form of the invention, the penetration-enhancing hydrolysable lipophilic excipient comprises at least one of a saturated fatty alcohol or fatty acid of 8-15 carbon atoms or of an unsaturated fatty alcohol or fatty acid of 8-18 carbon atoms.

And in one preferred form of the invention, the suitable pharmaceutical carrier comprises any non-toxic pharmaceutically suitable vehicle that comprises any polar protic solvent with a molecular weight of less than 600 (e.g., propylene glycol or polyethylene glycol).

### EXAMPLE

### Hyaluronic Acid Hydrogel Preparation

Formulations of taurolidine in aqueous solutions of hyaluronic acid (HA) crosslinked with 1,4-butanediol diglycidyl ether (BDDE) were prepared. 3% taurolidine were formulated in aqueous solutions of crosslinked HA of three molecular weights: low molecular weight (LMW) 21-40 kDa, medium molecular weight (MMW) 310-450 kDa and high molecular weight (HMW) 750 kDa-1.0 MDa. Control formulations were prepared without addition of the taurolidine. 1.5% myristic acid was added to enhance the interaction with the explant. In Table 1, the compositions of each formulation are given.

### Biofilm Porcine Skin Explant Model

The ex vivo model of biofilm on porcine skin explants used in this study consisted of 12-mm biopsied explants (3-4 mm thick) prepared from freshly harvested, shaved and cleaned porcine skin obtained from a local abattoir (Chiefland Custom Meat, Trenton, Fla.). The mechanically created “wound bed” (3-mm high speed, round cutter bit; Dremel®, Robert Bosch Tool Corporation, Racine, Wis.) was 3 mm in diameter and approximately 1.5 mm in depth at the centre of each explant. The chlorine gas (45 minutes)-sterilised explants were placed on soft TSA plates containing 0.5% agar and 50 μg/ml gentamicin. The addition of 50 μg/ml gentamicin (^{˜}30× minimal inhibitory concentration) functions to limit bacterial growth to the explant and inhibits penetration of Pseudomonas aeruginosa PAO1 biofilm through the bottom of the explant for up to -6 days, depending on the thickness of the explant. The partial-thickness “wound bed” of the explants was inoculated with 10 μl early-logarithmic (log)-phase PAO1 suspension culture (106 CFU) and cultured at 37° C. with 5% CO2 and saturated humidity. Explants were transferred daily to fresh soft TSA plates containing 0.5% agar and antibiotic (to maintain moisture) until the desired biofilm maturity was achieved. They were submerged in TSB media containing 200 μg/ml gentamicin for 24 hours to kill planktonic PAO1 in studies used to assess antimicrobial efficacy of test agents specifically against the highly antibiotic tolerant biofilm subpopulation attached to the porcine explants, described in more complete detail below. For clarity, exposure times to the test agents were expressed in hours and the length of biofilm culture incubation prior to treatment was expressed in days.

The bacterial load of the explants was determined in each of the assays of this study as follows: each explant was aseptically placed into a 15 ml sterile tube (on ice) containing cold 7 ml sterile phosphate-buffered saline (PBS) with 5 μl/l Tween-80. The explants in the tubes were sonicated with a 23 kHz ultrasonic dismembrator (Model 100, Fisher Scientific, Pittsburgh, Pa.) probe for 30 seconds at approximately 20 Watts on ice, which liberated bacteria from the biofilm into the suspension. The setting on the dismembrator probe tip was adjusted to maintain the target watt output. The sonication probe was disinfected between samples using cold 70% ETOH and rinsed with cold sterile PBS (on ice). Serial dilutions of the bacterial suspension were plated in triplicate on TSA plates and incubated overnight at 37° C. with 5% CO2 and saturated humidity. Colonies were counted from the plates to determine the CFU/ml of the sonicated explant bacterial suspension.

### Assessment of The Efficacy of Antimicrobial

### Dressings Against PAO1 Biofilm

72-Hour Continuous Exposure.

Antimicrobial efficacy assays against mature PAO1 biofilm attached to the skin were performed with 72-hour continuous exposure. PAO1 biofilms cultured 3 days on porcine skin explants were transferred to sterile 24-well Microtiter™ plates and each explant was treated for 24 hours by submersion in 2 ml TSB media containing 200 μg/ml gentamicin. This level of antibiotic was used because it was capable of restraining the PAO1 biofilm to the surface of the explant. The media in the wells remained clear and no viable bacteria were detected in the media or the Microtiter™ wells during or after treatment of the explants. As stated previously, pre-treatment with high levels of antibiotics allows subsequent assessment of the antimicrobial efficacy of the dressing agents directly against the antibiotic tolerant biofilm subpopulation. The antibiotic pre-treated explants, containing only mature PAO1 biofilm, were each rinsed thrice with 2 ml of sterile PBS, washed in 2 ml PBS for 10 minutes and then rinsed thrice with 2 ml PBS to remove unattached bacteria. The rinsed biofilm explants were transferred to soft TSA plates containing 0.5% agar and 50 μg/ml gentamicin (three or four explants per plate).

The biofilm explants that were used to determine the “standard” baseline total microbial load were covered with sterile double distilled H2O-saturated (5 ml) “wet” cotton gauze sponge (2″×2″). The rest of the biofilm explants were covered and treated with 1 ml of Hyaluronic Acid loaded hydrogels as shown in Table 1. The treated biofilm explants were each processed by sonication in 7 ml PBS with 5 μl/l Tween-80, as previously described. Bacterial suspensions were immediately serially diluted and plated in triplicate on TSB, and the average CFU/ml was determined for the 7 ml bacterial suspension from each explant. A minimum of three separate trials were performed for each antimicrobial dressing reported in this study.

### Time-Course Assay

The time-course studies were performed to determine the antimicrobial efficacy of the taurolidine hydrogels on biofilm maturity. The biofilm explants were continuously exposed to dressing for 72 hours. The treated explants were each processed by sonication in 7 ml PBS with 5 μl/l Tween-80 as previously described. Bacterial suspensions were immediately serially diluted and plated in triplicate on TSB, and the average CFU/ml was determined for the 7 ml bacterial suspension from each explant.

6 samples from Cambridge Polymer Group

Day 0: PA01 OD600=0.243 Concentration=1.21E08 cells/ml

Day 3: put 3 day cultured explants in 24 well treat with 1 ml different solution.

Day 4: cell count.

TABLE 1

| | AVG | |
|---|---|---|
| PA01 | (cells/ml) | STDEV |
| Total(3 day cultured PA01 explants) | 1.47E+09 | 1.43E+08 |
| Biofilm, 200 ug/ml Gentamicin | 3.45E+07 | 4.68E+07 |
| 13146-1, LMW HA control(no drug), | 9.32E+06 | 4.12E+06 |
| 1.5% Myristic acid | | |
| 13146-2, MMW HA control(no drug), | 4.18E+07 | 3.65E+07 |
| 1.5% Myristic acid | | |
| 13146-3, HMW HA control(no drug), | 5.78E+07 | 6.60E+07 |
| 1.5% Myristic acid | | |
| 13146-4, LMW HA, 3% drug, | 7.22E+01 | 1.03E+02 |
| 1.5% Myristic acid | | |
| 13146-5, MMW HA 3% drug, | 4.44E+01 | 7.70E+01 |
| 1.5% Myristic acid | | |
| 13146-6, , HMW HA 3% drug, | 0.00E+00 | 0.00E+00 |
| 1.5% Myristic acid | | |

These results show that taurolidine-loaded hydrogels effectively penetrate and break-up the biofilm and kill biofilm embedded microorganisms such as Pseudomonas aeruginosa (PA01).

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

1. (canceled)

2. A method according to claim 22 wherein the hydrolysable taurolidine is selected from the group consisting of taurolidine and a salt thereof.

3. A method according to claim 22 wherein the hydrolysable lipophilic excipient comprises at least one of a saturated fatty alcohol or fatty acid of 8-15 carbon atoms.

4. A method according to claim 22 wherein the hydrolysable lipophilic excipient comprises at least one of an unsaturated fatty alcohol or fatty acid of 8-18 carbon atoms.

5. A method according to claim 22 wherein the hydrolysable lipophilic excipient comprises at least one of myristic acid and myristyl alcohol.

6. A method according to claim 22 wherein the hydrolysable lipophilic excipient comprises small peptides provided with lipophilic side chains.

7. A method according to claim 6 wherein the small peptides have a high percentage of valine, leucine, proline, phenylalanine, tryptophan and/or leucine-enkephalin.

8. A method according to claim 6 wherein the hydrolysable lipophilic excipient comprises fatty acid esters.

9. A method according to claim 8 wherein the fatty acid esters include 10-15 carbon saturated and unsaturated fatty esters.

10. A method according to claim 9 wherein the fatty acid esters include compositions comprising diglycerides, triglycerides, and glycerol monostearate.

11. (canceled)

12. A method according to claim 22 wherein the active moieties comprise methylol groups.

13. A method according to claim 22 wherein the hydrolysable taurolidine is mixed into a mass of the hydrolysable lipophilic excipient.

14. A method according to claim 22 wherein the hydrolysable taurolidine and the hydrolysable lipophilic excipient are in the form of nanoparticles, wherein the hydrolysable taurolidine comprises a core and the hydrolysable lipophilic excipient comprises an encapsulating cover over the hydrolysable taurolidine core.

15. A method according to claim 22 wherein the hydrolysable taurolidine and the hydrolysable lipophilic excipient are suspended in an emulsion.

16. A method according to claim 15 wherein the emulsion comprises a polar protic solvent with a molecular weight of less than 600.

17. A method according to claim 15 wherein the emulsion comprises at least one of propylene glycol, polyethylene glycol, petrolatum, glycerin, polyvinylpyrrolidone and hyaluronic acid.

18. A method according to claim 22 wherein the composition further comprises a suitable pharmaceutical carrier.

19. (canceled)

20. A method according to claim 18 wherein the pharmaceutical carrier comprises a non-toxic pharmaceutically-suitable vehicle which comprises any polar protic solvent with a molecular weight of less than 600.

21. A method according to claim 20 wherein the pharmaceutical carrier comprises at least one from the group consisting of propylene glycol and polyethylene glycol.

22. A method for treating a patient, the method comprising:
applying a composition to the skin of a patient, the composition comprising:
hydrolysable taurolidine; and
a hydrolysable lipophilic excipient;
wherein the hydrolysable taurolidine is contained within the hydrolysable lipophilic excipient; and
leaving the composition on the skin of the patient long enough for the hydrolysable lipophilic excipient to facilitate passage of the composition through the skin and, as the composition passes through the skin, the lipophilic excipient is hydrolyzed, exposing the hydrolysable taurolidine to the anatomy, whereupon the taurolidine hydrolyzes into its active moieties so as to provide local antimicrobial effects.
